(19) United States
(12) Patent Application Publication
(10) Pub. No.: US 2015/0225314 A1
(43) Pub. Date: Aug. 13, 2015

(54) METHOD FOR THE MANUFACTURE OF AROMATIC HYDROCARBONS

(71) Applicant: Teknologian Tutkimuskeskus VTT Oy, Vtt (FI)

(72) Inventors: Martta Asikainen, Vtt (FI); Olli Aaltonen, Vtt (FI); Ali Harlin, Vtt (FI); Tiina Nakari-Setala, Vtt (FI)

(21) Appl. No.: 14/428,165

(22) PCT Filed: Sep. 13, 2013
(86) PCT No.: PCT/FI2013/050888

§ 371 Date: Mar. 13, 2015

(30) Foreign Application Priority Data
Sep. 14, 2012 (FI) 20125950

(51) Int. Cl. C07C 5/48

(52) U.S. Cl. CPC C07C 5/48

(57) ABSTRACT

A method for the manufacture of aromatic hydrocarbons, includes allowing at least one olefin to react with at least one hydrogen scavenger at a temperature from 50 to 1000° C., under a pressure from normal atmospheric pressure to 500 bar without an added catalyst, to yield aromatic hydrocarbons.

### FIELD OF THE INVENTION

The present invention relates to a method for the manufacture of aromatic hydrocarbons from olefins. The invention also relates to a method for the manufacture of paracymene. Further, the invention relates to a method for the manufacture of aromatic hydrocarbons from renewable starting materials.

### BACKGROUND

In the field of aromatic compounds there is an increasing interest in providing more efficient and economic methods for their manufacture. Also, industrially feasible methods utilizing starting materials based on renewable sources, for the manufacture of aromatics, are currently sought. Aromatics are widely used in the chemical industry, for example as starting materials in the manufacture of intermediates and polymers. An example of such intermediate is terephthalic acid, which is currently manufactured from paraxylene. It may, however, also be manufactured from paracymene.

Paracymene (1-methyl-4-propan-2-ylbenzene) is an aromatic hydrocarbon, which can be used for example in the manufacture of polyester plastics, particularly polyetheneterephthalate (PET) and antioxidants, such as butylhydroxytoluene (BHT). PET is very widely used industrial plastic with high production volumes, for example in bottles and textile fibers.

Paracymene is spontaneously formed from a simple monoterpene γ-terpinene. Compt. Rend. (1964), 258(22), 5539-41 describes spontaneous and gradual formation of paracymene under air from γ-terpinene isolated from Thymus vulgaris. The spontaneous oxidation leading to aromatization is slow and non-selective. Therefore this synthesis, which occurs in nature, cannot be readily transformed to industrial practice.

Many alternative routes for the manufacture of paracymene have been developed, comprising several stages and typically requiring catalytic reaction steps.

Terpinene is a cyclic olefin belonging to the group of monoterpenes. It has been used as starting material for the manufacture of paracymene, typically in the presence of exotic catalysts, enzymes and other reagents, as well as strong oxidizing agents. Said methods often comprise several steps.

WO 2012006039 discloses several alternative methods for the manufacture of aromatic compound from renewable sources. Said methods are based on the dehydrogenation of cyclic monoterpenes using as dehydrogenation catalysts metal catalysts, zeolites, acid catalysts and enzymes.

A method comprising two synthesis stages, for the manufacture of paracymene is described in WO 2010078328 where a terpene, terpenoid or a mixture thereof is separated from biomass, followed by converting it to paracymene in the presence of a catalyst selected from metal catalysts, amine catalysts and combinations thereof. The bio-based paracymene is then converted to terephthalic acid by oxidation.

J. Phys. Org. Chem., 2003, 16, 16-20 teaches the synthesis of paracymene from monocyclic, bicyclic or acyclic monoterpenes using a thionine catalyst on a Na—Y zeolite carrier. This catalytic aromatization method yields at most 36% by weight of an aromatic product.

Solvent-free dehydrogenation of γ-terpinene in a ball mill with finely divided alumina, quartz or montmorillonite, in the presence of an oxidizing agent (KMnO₄, ozone, NaIO₄, or I₂) is presented in Green Chem., 2010, 12(7), 1288-94.

Publication Applied Catalysis A: General 351, 2008, 226-239 describes aerobic dehydrogenation of terpines in the presence of p-benzoquinone catalyst, with acetic acid as solvent. Cu(OAc)₂ was used as a co-catalyst for improving dehydrogenation.

J. Org. Chem. 1989, 54, 4607-4610 describes the heteropoly acid catalyzed aromatization of 1,2-dihydronaphthalene to naphthalene under oxygen.

Eur. J. Inorg. Chem. 2003, 3539-3546 describes the ruthenium-catalyzed aromatization of 1,2-dihydronaphthalene to naphthalene.

Tet. Lett. 2010, 51, 1822-1825 describes the copper-catalyzed (CuCl—PPh3) aromatization of 1,2-dihydronaphthalene to naphthalene.

Based on the above it can be seen that there exists a need to provide new and improved methods for the manufacture of aromatic hydrocarbons, as well as for industrially feasible methods utilizing starting materials based on renewable sources for the manufacture of aromatics, for use in the chemical industry as such, and as starting materials and intermediates for the manufacture of other compounds.

### SUMMARY

An object of the invention was to provide an improved method for the manufacture of aromatic hydrocarbons from olefins.

Another object of the invention was to provide an improved method for the manufacture of aromatic hydrocarbons from olefins, where said olefins are based on renewable materials.

A further object of the invention was to provide an improved method for the manufacture of paracymene from olefins.

A still further object of the invention was to provide an improved method for the manufacture of paracymene from renewable materials.

The present invention relates to a method for the manufacture of aromatic hydrocarbons from olefins. The invention also relates to a method for the manufacture of aromatic hydrocarbons from renewable starting materials. The invention further relates to a method for the manufacture of paracymene, particularly from renewable starting materials.

It was surprisingly found that it is possible to increase the aromatization rate tremendously, without losing selectivity, by elevating the reaction temperature. A catalyst is not needed if aromatization is carried out in the presence of a hydrogen scavenger, under suitable pressures. Allowing suitable residence time for the reaction mixture is also advantageous.

This finding is contrary to what is known about the effect of increasing temperature on aromatization selectivity. Choudhary and Devadas (Microporous and Mesoporous materials, Vol. 23, Issues 3-4, p 231-238), for example, report their findings that the aromatization activity of their catalyst was markedly decreased with the increase of temperature.

The method for the manufacture of aromatic hydrocarbons according to the invention comprises the steps where at least one olefin is allowed to react with at least one hydrogen scavenger at the temperature of 50-1000° C., under a pressure from normal atmospheric pressure to 500 bar without an added catalyst, to yield aromatic hydrocarbons.

Said olefin may be selected from cyclic olefins, branched olefins and linear olefins, containing at least one double bond. Examples of suitable olefins are terpenes and terpinenes, which may be obtained from renewable materials or sources. However, suitable olefins may also be of synthetic origin, obtained from non-renewable sources and/or from other processes.

The method for the manufacture of paracymene comprises the steps where at least one olefin is allowed to react with at least one hydrogen scavenger at the temperature of 50-1000° C., under a pressure from normal atmospheric pressure to 500 bar without an added catalyst, to yield paracymene.

The characteristic features of the invention are presented in the appended claims.

### DEFINITIONS

Unless otherwise specified, the terms, which are used in the specification and in the claims, have the meanings commonly used in the field of organic chemistry. Specifically, the following terms have the meanings indicated below.

The term “renewable” means biological material derived from living or recently living organisms or part of it. Renewable is distinguished from non-renewable, fossil-derived matter.

The term “normal atmospheric pressure” refers here to the pressure at any location on the earth, caused by the weight of the column of air above it. At sea level, normal atmospheric pressure has an average value of one atmosphere (1 kg/cm²).

Unless otherwise noted, all percentages are by weight.

### BRIEF DESCRIPTION OF THE FIGURES

FIG. 1 illustrates a GC-MS chromatogram of 4 h sample, 15.49 min (paracymene) and 19.18 min (γ-terpinene) from Example 1, Run 4.

FIG. 2 illustrates a GC-MS chromatogram of 6 h sample, 15.49 min (paracymene) and 19.18 min (γ-terpinene) from Example 1, Run 4.

FIG. 3 illustrates a GC-MS chromatogram of the product, 15.49 min (paracymene) from Example 2.1.

FIG. 4 illustrates a GC-MS chromatogram of the 4 h sample, 15.49 min (paracymene) and 14.75 min (α-terpinene) from Example 3, Run 2.

FIG. 5 illustrates a GC-MS chromatogram of the 4 h sample, 15.49 min (paracymene) and 13.71 min (α-phellandrene) from Example 3, Run 3.

FIG. 6 illustrates a GC-MS chromatogram of the product, 31.51 min (1,2-dihydronaphthalene) and 33.14 min (naphthalene) from Example 4.

### DETAILED DESCRIPTION OF THE INVENTION

The present invention is based on studies relating to dehydroaromatization of olefins, particularly of terpenes, such as terpinenes. The olefin, such as terpinenes may be obtained from renewable materials. However, olefins may also be of synthetic origin obtained from other sources and/or from other processes.

Non-limiting examples of renewable olefins include monoterpenes, monoterpenoids and related compounds produced by a large variety of living organisms.

Methods for isolating cyclic monoterpenes are generally known to those of skill in the art.

According to the method of the invention, aromatic hydrocarbons can be obtained from olefins with excellent yields and selectively without any added catalyst whereby the aromatic compound can be produced more efficiently and economically. The reaction is surprisingly fast and selective. For example paracymene can be obtained from terpinenes, whereby a product based at least partly or even totally on renewable sources can be obtained.

In the method a hydrogen scavenger, such as oxygen or oxygen containing gas (gas mixture containing oxygen) is brought into contact with the olefin, suitably at an elevated temperature, where the hydrogen scavenger deprives one hydrogen molecule from the olefin (such as terpinene C6-ring) and forms water with it. Oxygen acts here as a selective hydrogen scavenger and practically no side-products besides water are produced.

The method according to the invention, for the manufacture of aromatic hydrocarbons comprises the steps where at least one olefin is allowed to react with at least one hydrogen scavenger at the temperature from 50 to 1000° C., under a pressure from normal atmospheric pressure (1 bar=100 KPa) to 500 bar (50000 KPa) without an added catalyst, to yield aromatic hydrocarbons.

The olefin is selected from cyclic olefins, branched olefins and linear olefins, containing at least one double bond, and from mixtures thereof. Cyclic olefins refer here to monocyclic and polycyclic olefins. The cyclic olefin contains preferably at least one C6 ring (contains 6 carbon atoms). Preferably each ring contains at least one double bond. Preferably cyclic olefins are used, and suitably cyclic olefins are selected from monoterpenes and terpinenes.

Examples of suitable olefins are listed as follows: α-terpinene, β-terpinene, γ-terpinene, δ-terpinene, α-phellandrene, β-phellandrene, cineole, camphene, and 1,2-dihydronaphthalene.

Aromatization of some suitable olefins is presented in following schemes 1-4:

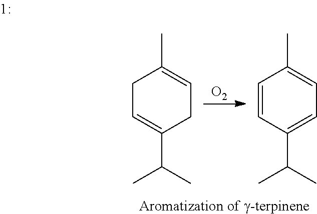

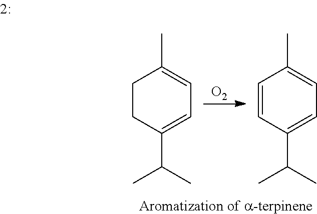

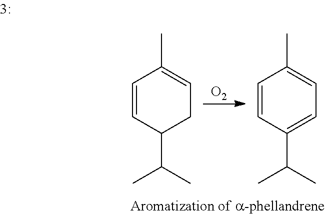

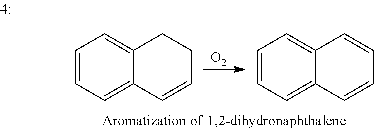

The hydrogen scavenger is selected from oxygen, oxygen containing gas mixtures, such as air, synthetic air, and mixtures of oxygen with one or more inert gases, such as nitrogen, carbon dioxide, noble gases. The oxygen content may be adjusted in the method to provide at least ½ mole of O₂ with respect to each mole of leaving H₂ from the olefin.

The temperature is preferably from 70 to 400° C., particularly preferably from 100 to 300° C.

The pressure is preferably from normal atmospheric pressure to 100 bar.

The reaction may be carried out in one phase: in liquid phase or in gas phase or in vapor phase, preferably vapor phase is used. Alternatively the reaction may be carried out as a two-phase reaction.

The method may be carried out as a batch process, semi-continuous process or continuous process.

The residence time may range from 1 s to 50 h, in a continuous process it is typically from 1 s to 10 min, in batch process from 30 min to 50 h.

Optionally one or more solvents may be used in the reaction, and suitably inert solvents having high boiling points, such as aromatic solvents and chlorinated aromatic solvents may be used.

Suitably any mixing tank or reactor may be used for the method, equipped with means to provide efficient/vigorous agitation.

If desired, any known separation, fractionation, crystallization, purification or work-up procedures may be used.

The method according to the invention has several advantages.

The use of catalysts in the dehydroaromatization reaction can be avoided, and thus no purification steps for removing catalyst poisons are needed. The dehydroaromatization reaction with oxygen is very exothermic. In catalyzed reactions there is a risk of hot spot formation within the catalyst bed, which leads to coke formation, loss of catalyst activity and selectivity and loss of yield. In the method according to the invention hot spot formation in the catalyst bed is obviously avoided. The method according to the invention gives a much wider operating window than the methods which use a solid catalyst. The purity of the feedstock does not need to be as high as with catalyzed methods. The process parameters (temperature, oxygen concentration, pressure, residence time) can be more freely adjusted without the risk of causing catalyst deactivation and loss of selectivity and yield. A very simple hydrogen scavenger, such as air or oxygen containing gas can be used, preferably in combination with elevated temperature, suitable pressure and suitable residence time. This provides good reaction rate, excellent yields with high selectivity and minimum amount of side-reactions.

The aromatic hydrocarbons obtained with the method of the invention may be used in various applications in the chemical industry. Suitably they may be utilized as starting materials and intermediates in the manufacture valuable compounds, such as terephthalic acid, and in the manufacture of polymers and antioxidants.

The invention also provides a method for the manufacture of paracymene. If desired, paracymene may be manufactured from renewable starting materials, to yield at least partly or totally bio-based paracymene, which may be used as starting material for the manufacture of various products, such as bio-based polymers and antioxidants. The following examples are illustrative of embodiments of the present invention, as described above, and they are not meant to limit the invention in any way.

### EXAMPLES

### Example 1

### Continuous Aromatization of γ-Terpinene

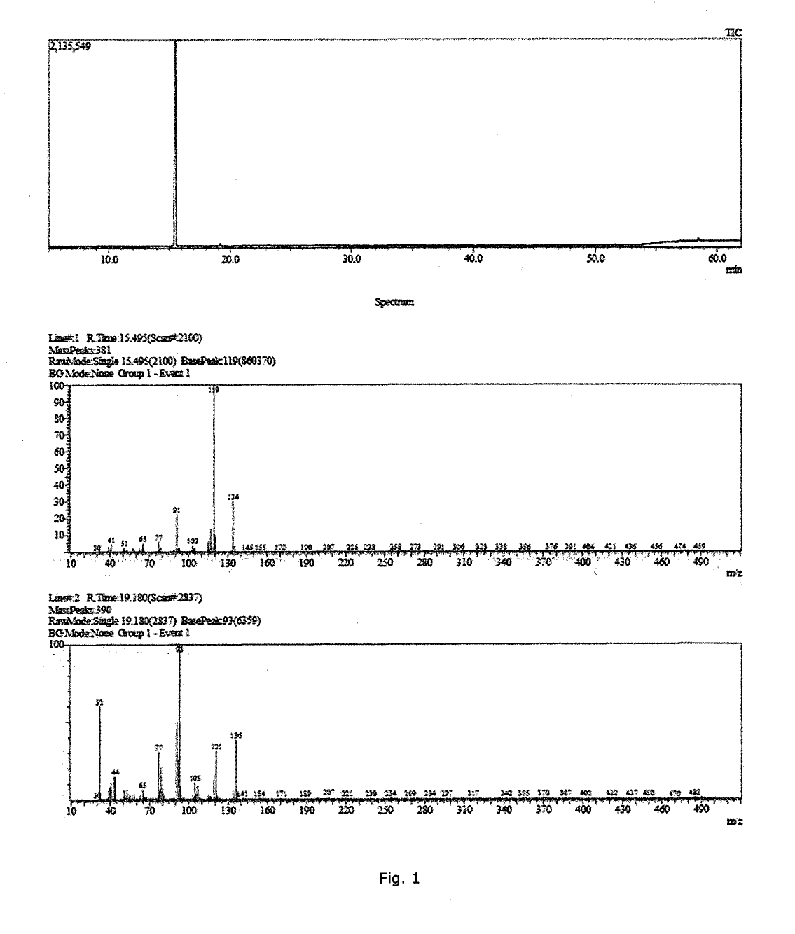

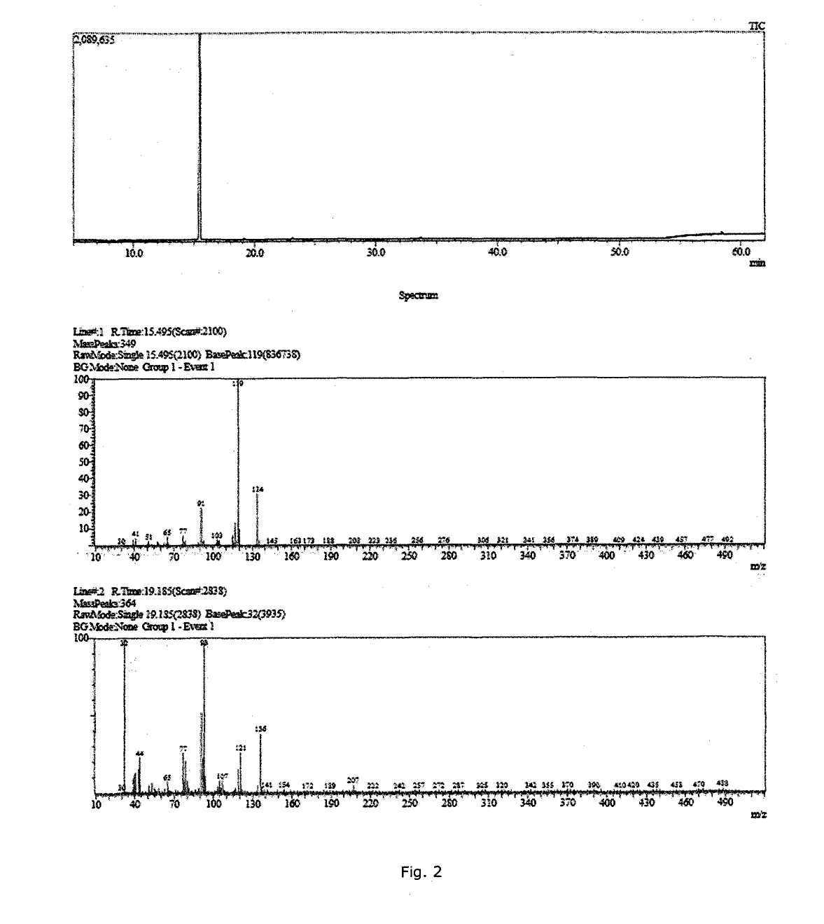

A continuously operating reactor was heated to the reaction temperature and pressurized with carrier gas. γ-terpinene (assay >95%) was fed to the reactor with 4.5 g/h feed with 3.28 l/h carrier gas. The collecting flask was cooled to +5° C. and samples of the product mixture were collected every two hours. The organic phases of the product fractions were separated, weighed and analysed with GC and GC-MS. In GC analyses n-dodecane was used as internal standard. In FIG. 1 a GC-MS chromatogram of 4 h sample of Run 4, 15.49 min (paracymene) and 19.18 min (γ-terpinene) is presented. In FIG. 2 a GC-MS chromatogram of 6 h sample of Run 4, 15.49 min (paracymene) and 19.18 min (γ-terpinene) is presented. The results are presented in following Table 1.

TABLE 1

| | | | | p-cymene | p-cymene | Organic |
|---|---|---|---|---|---|---|
| | T | P | Carrier | 4 h | 6 h | fraction |
| Run | (° C.) | (bar) | gas^{a)} | (wt-% GC) | (wt-% GC) | yield (wt-%) |
| 1 | 200 | 10 | Air | 86 | 82 | 85 |
| 2 | 200 | 10 | Argon | 5 | 3 | 92 |
| 3 | 200 | 10 | O₂/ | 71 | 79 | 85 |
| | | | Argon^{b)} | | | |
| 4 | 250 | 30 | Air | >95 | >95 | 82 |
| 5 | 150 | 75 | Air | 40 | 38 | 90 |

^{a)}Synthetic air contains 20% O₂ and 80% N₂
^{b)}O₂ 1.64 l/h and argon 1.64 l/h

### Example 2

### Aromatization of γ-Terpinene in a Batch Reactor

2.1

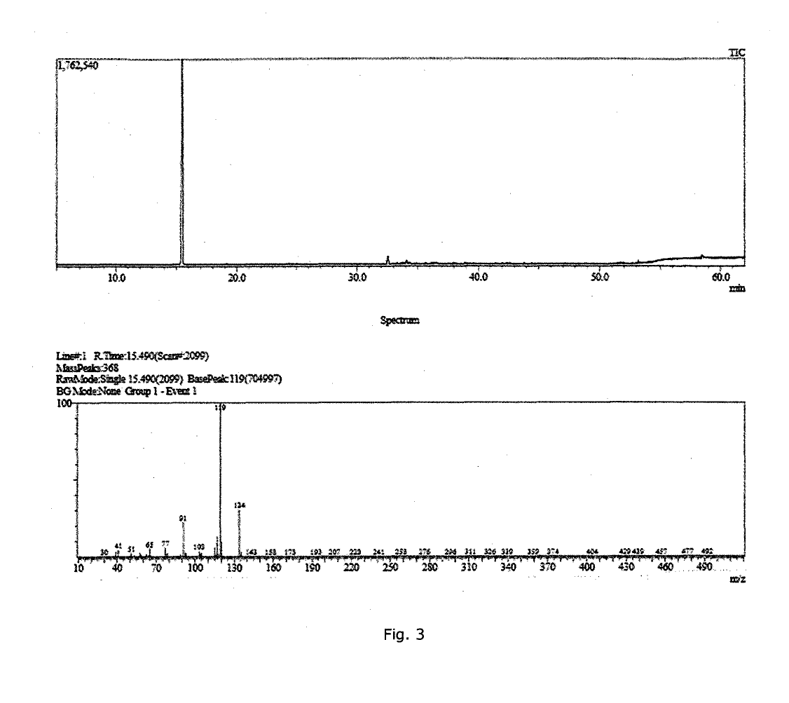

To a glass reactor fitted with a Dean-Stark water separator, mechanical stirrer, reflux condenser and pipe for bubbling gas (air) was charged γ-terpinene (405 g, assay >95%). The reaction mixture was heated at 125° C. for 27 h, air was bubbled through the reaction mixture and stirring was maintained at 400 rpm. The product (327 g) was obtained as clear yellow liquid. According to GC analysis the product contained 94% by weight of paracymene, no starting material could be detected. A GC-MS chromatogram of the product, 15.49 min (paracymene) is presented in FIG. 3.

2.2

To a glass reactor fitted with a Dean-Stark water separator, mechanical stirrer, reflux condenser and pipe for bubbling gas (air) was charged y-terpinene (33.3 g, assay >95%) and toluene (34.1 g). The reaction mixture was heated to 110° C. for 4 h, air was bubbled to the reaction mixture and stirring was maintained at 400 rpm. The solvents were removed by distillation and the residue (33.0 g) contained 9.5% by weight paracymene and 84% by weight γ-terpinene.

2.3

To a Teflon coated pressure reactor was charged y-terpinene (1.0 g, assay >95%). The reactor was heated to 160° C. and pressurized to 15 bar with synthetic air (20% O₂ and 80% N₂) for 18 hours. The product (0.62 g) contained 80% by weight paracymene and no starting material could be detected.

### Example 3

### Continuous Aromatization of Other Terpenes

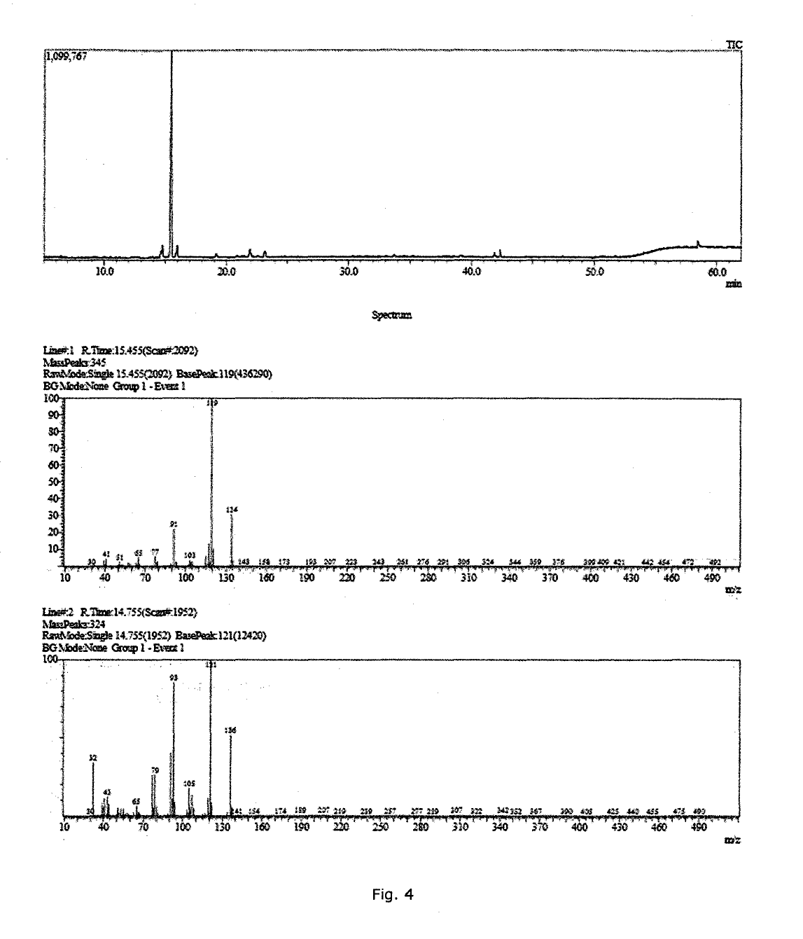

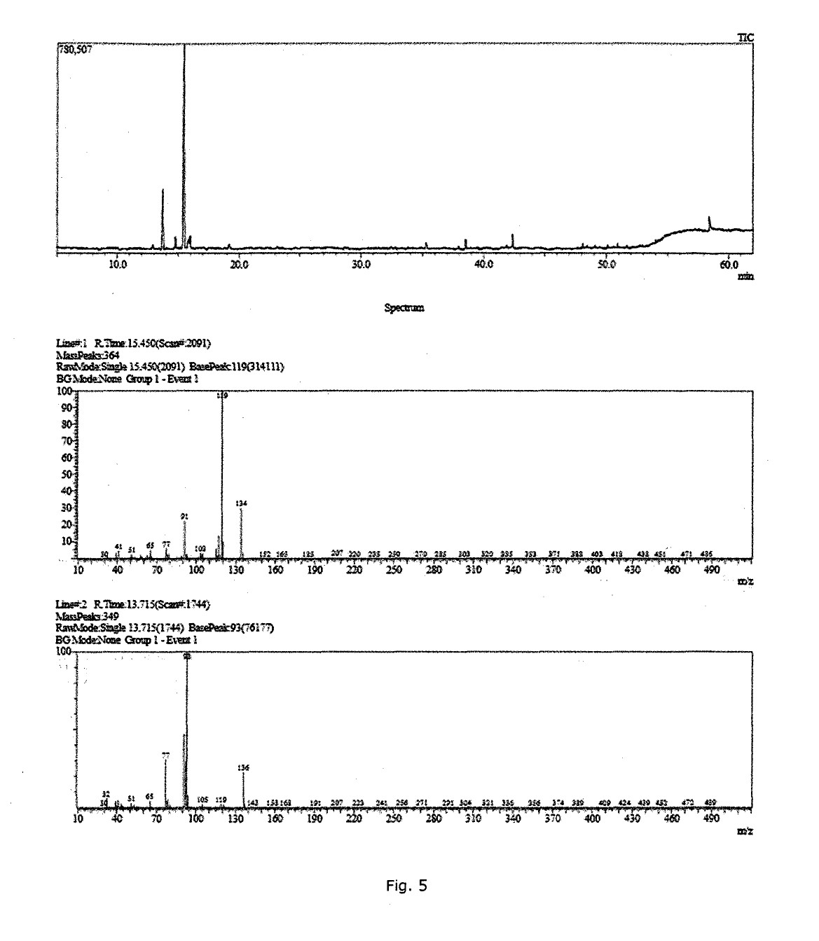

Continuously operating reactor was heated to the reaction temperature and pressurized with carrier gas. Terpene feed was fed to the reactor with 4.5 g/h feed with 3.28 l/h carrier gas (synthetic air containing 20% O₂, 80% N₂). The collecting flask was cooled to +5° C. and samples of the product mixture were collected every two hours. The organic phases of the product fractions were separated, weighed and analysed with GC and GC-MS. In GC analyses n-dodecane was used as internal standard. A GC-MS chromatogram of the 4 hour sample, Run 2, 15.49 min (paracymene) and 14.75 min (α-terpinene) is presented in FIG. 4. A GC-MS chromatogram of the 4 hour sample, Run 3, 15.49 min (paracymene) and 13.71 min (α-phellandrene) is presented in FIG. 5. The results are presented in Table 2.

TABLE 2
Examples with other terpenes

| | | | p-cymene | p-cymene | Organic |
|---|---|---|---|---|---|
| | | Condi- | 4 h | 6 h | fraction |
| Run | Feed | tions^{a)} | (wt-% GC) | (wt-% GC) | yield (wt-%) |
| 1 | α- | A | 38 | 42 | 94.9 |
| | Terpinene | | | | |
| 2 | α- | B | 43 | 48 | 81.6 |
| | Terpinene | | | | |
| 3 | α- | A | 31 | 26 | 88.0 |
| | Phellandrene | | | | |

^{a)}Reaction conditions A: 200° C., 10 bar synthetic air (20% O₂, 80% N₂), Reaction conditions B: 250° C., 30 bar synthetic air (20% O₂, 80% N₂)

### Example 4

### Aromatization of 1,2-Dihydronaphthalene

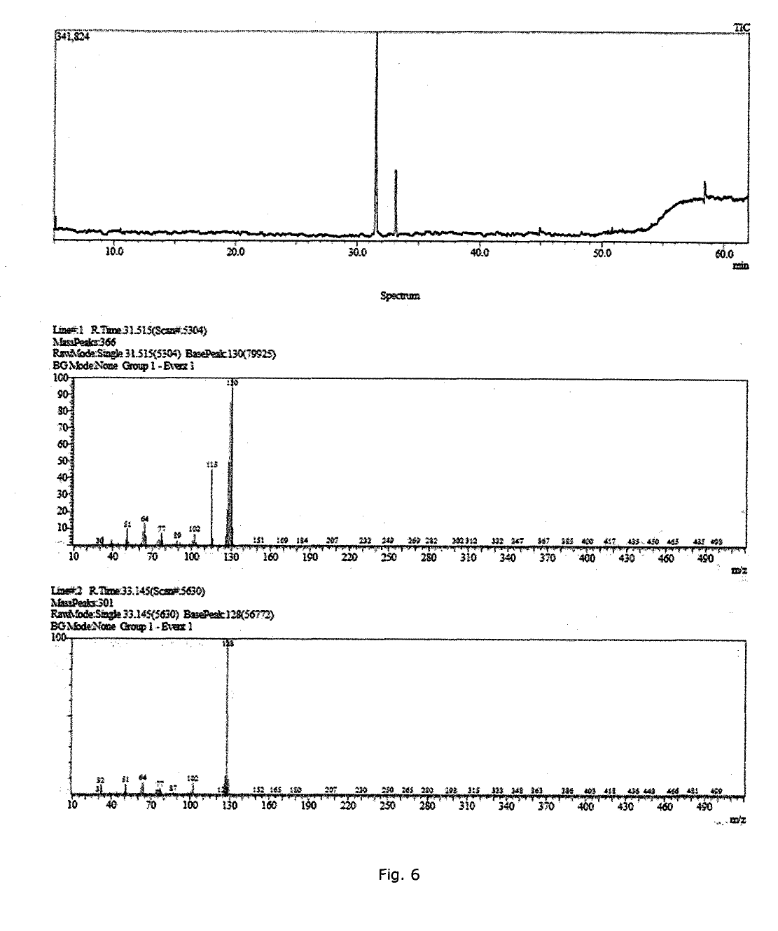

To a Teflon coated pressure reactor was charged 1,2-dihydronaphthalene (0.24 g, assay 99%) and toluene (2.51 g). The reactor was heated to 190° C. and pressurized to 15 bar with synthetic air (20% O₂ and 80% N₂) for 4 hours. The product (0.21 g) contained 22% by weight naphthalene and 77% by weight 1,2-dihydronaphthalene. A GC-MS chromatogram of the product, 31.51 min (1,2-dihydronaphthalene) and 33.14 min (naphthalene) is presented in FIG. 6.

1. A method for the manufacture of aromatic hydrocarbons, characterized in that the method comprises the steps where at least one olefin is allowed to react with at least one hydrogen scavenger selected from oxygen and gas mixtures containing oxygen, at the temperature from 50 to 1000° C., under a pressure from normal atmospheric pressure to 500 bar without an added catalyst, and the oxygen content is adjusted to provide at least mole of O₂ with respect to each mole of leaving H₂ from the olefin, to yield aromatic hydrocarbons.

2. The method according to claim 1, characterized in that the olefin is selected from cyclic olefins, branched olefins and linear olefins, containing at least one double bond, and mixtures thereof.

3. The method according to claim 1, characterized in that the cyclic olefin contains at least one C6 ring and each ring contains at least one double bond.

4. The method according to claim 1, characterized in that the cyclic olefin is a monoterpene or terpinene, preferably selected from α-terpinene, β-terpinene, γ-terpinene, δ-terpinene, α-phellandrene, β-phellandrene, cineole, camphene, and 1,2-dihydronaphthalene.

5. The method according to claim 1, characterized in that the temperature is from 70 to 400° C., preferably from 100 to 300° C.

6. The method according to claim 1, characterized in that the pressure is from 5 to 100 bar.

7. The method according to claim 1, characterized in that the reaction is carried out as one phase reaction or as two-phase reaction.

8. The method according to claim 1, characterized in that the reaction is carried out in liquid phase or in gas phase or in vapor phase.

9. The method according to claim 1, characterized in that the method is carried out as batch process, semi-continuous process or continuous process.

10. The method according to claim 1, characterized in that the residence time ranges from 1 s to 50 h, in a continuous process preferably from 1 s to 10 min, and in batch process preferably from 30 min to 50 h.

11. The method according to claim 1, characterized in that the aromatic hydrocarbon is paracymene.

12. The method according to claim 1, characterized in that the olefin originates from renewable sources.

13. The method according to claim 2, characterized in that the cyclic olefin contains at least one C6 ring and each ring contains at least one double bond.
